(19) United States
(12) Patent Application Publication
(10) Pub. No.: US 2010/0234503 A1
(43) Pub. Date: Sep. 16, 2010

(54) POLYMER COMPOSITES MECHANICALLY REINFORCED WITH ALKYL AND UREA FUNCTIONALIZED NANOTUBES

(76) Inventors: Valery N. Khabashesku, Houston, TX (US); Merlyn X. Pulikkathara, Houston, TX (US)

Correspondence Address: WINSTEAD PC, P.O. BOX 50784, DALLAS, TX, 75201, US

(21) Appl. No.: 12/376,901

(22) PCT Filed: Aug. 10, 2007
(86) PCT No.: PCT/US07/75727

§ 371 Date: May 24, 2010

Related U.S. Application Data
(60) Provisional application No. 60/837,016, filed on Aug. 10, 2006.

(51) Int. Cl. C08K 5/03; C08F 10/02; C07F 7/10; C07C 335/00

(52) U.S. Cl. 524/213; 524/462; 525/333.7; 556/421; 564/29; 564/55; 977/742; 977/753; 977/750; 977/746

(57) ABSTRACT

A polymer composite includes a polymer matrix and an alkyl-substituted carbon nanotube. A polymer composite also includes a polymer matrix and a fluorinated carbon nanotube reacted with urea, thiourea, or guanidine. A method of functionalizing a carbon nanotube includes heating a fluorinated carbon nanotube urea, thiourea, or guanidine. A substituted carbon nanotube includes a fluorinated carbon nanotube and amino silane compounds The amino silane compounds covalently link to the fluorinated nanotube through the amino functional group. Polymer composites, ceramics and surface coating materials may be constructed from these substituted carbon nanotubes.

### CROSS-REFERENCE TO RELATED APPLICATIONS

This application claims priority to U.S. Provisional Application No. 60/837,016, filed Aug. 10, 2006 and is incorporated herein by reference in its entirety.

### STATEMENT OF FEDERALLY-SPONSORED RESEARCH

This work was supported, in part, by NASA in the form of a Harriett G. Jenkins Pre-Doctoral Fellowship, by the U.S. Civilian Research and Development Foundation (CRDF) and by Air Force Research Laboratory Contract FA8650-05-D-1912.

### TECHNICAL FIELD

The present invention relates generally to nanostructured materials and specifically to functionalized carbon nanotubes in thermoplastic and thermoset composites.

### BACKGROUND INFORMATION

Single-wall carbon nanotubes (SWNTs) have highly anisotropic mechanical properties, however, by processing fully integrated single-walled carbon nanotube composites into nanotube continuous fibers (NCFs), their highly directional properties can be more effectively exploited. Manipulating these nanoscopic materials into an aligned configuration can be accomplished more easily by processing the composites into fibers, allowing for better macroscopic handling of these nano-sized materials. In some cases, the SWNTs have been used as nanoscale reinforcements in a polymer matrix in order to take advantage of their high elastic modulus (approaching 1 TPa) and tensile strengths (in the range 20-200 GPa for individual nanotubes). SWNTs are, however, more likely to be incorporated in the matrix as ropes or bundles of nanotubes, as a result of van der Waals forces that hold many entangled ropes together. These ropes or bundles have tensile strengths in the range of 15-52 GPa.

Polypropylene is an exemplary thermoplastic material that has excellent chemical resistance, and good mechanical properties with tensile strengths in the range of 30-38 MPa and tensile modulii ranging from 1.1-1.6 GPa for the bulk material. SWNTs incorporated into polypropylene matrices can result in a 40% increase in fiber tensile strength for composites containing a 1 wt. % loading of SWNTs by weight, although not necessarily displaying any significant improvements in other mechanical properties. It has been suggested that the efficient load transfer between the polymer matrix and the stronger, reinforcing SWNTs is not necessarily achieved.

In processing CNTs and a thermoplastic matrix into a fully integrated composite system, the chemically inert nature of each of these materials must be overcome in order to facilitate good interfacial adhesion, which in turn allows for better load transfer when a tensile load is applied to the system. Ineffective interfacial bonding, and sliding of individual nanotubes within nanotube ropes, will hamper load transfer from the matrix to the fiber, thereby limiting the amount of mechanical reinforcement that can be achieved in the composite.

As a result of the foregoing, a method for enhancing interfacial adhesion between the carbon nanotubes and the surrounding polymer matrix in composite materials would be quite beneficial.

### BRIEF SUMMARY OF THE INVENTION

In some aspects, the present disclosure provides a polymer composite that includes a polymer matrix and an alkyl-substituted carbon nanotube. In other aspects, the present disclosure provides a polymer composite that includes a polymer matrix and a fluorinated carbon nanotube reacted with a compound of formula I:

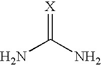

wherein X is selected from the group consisting of O, S, and NH.

In yet another aspect, the present disclosure provides a method of functionalizing a carbon nanotube that includes heating a fluorinated carbon nanotube with a compound of formula I:

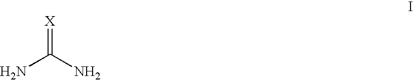

wherein X is selected from the group consisting of O, S, and NH.
In still further aspects, the present invention provides a substituted carbon nanotube that includes a fluorinated carbon nanotube and a compound of formula II:

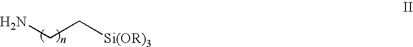

wherein n is an integer from 0 to 10; and R is an optionally-substituted alkyl group. The compound of formula II is covalently attached to the fluorinated nanotube through the amino functional group. Polymer composites, ceramics and surface coating materials may be constructed from these substituted carbon nanotubes.

The foregoing has outlined the features and technical advantages of the present invention in order that the detailed description of the invention that follows may be better understood. Additional features and advantages of the invention will be described hereinafter which form the subject of the claims of the invention.

### BRIEF DESCRIPTION OF THE DRAWINGS

The foregoing and other features and aspects of the present invention will be best understood with reference to the following detailed description of a specific embodiment of the invention, when read in conjunction with the accompanying drawings, wherein:

FIG. 1 shows Raman spectra of pristine SWNTs, trace A, phenylated SWNTs 1a, trace B, and phenylated SWNTs 1a after TGA, trace C.

FIG. 2 shows UV-Vis-NIR spectra showing a comparison between unfunctionalized SWNTs, trace A, and phenylated SWNTS 1a, trace B.

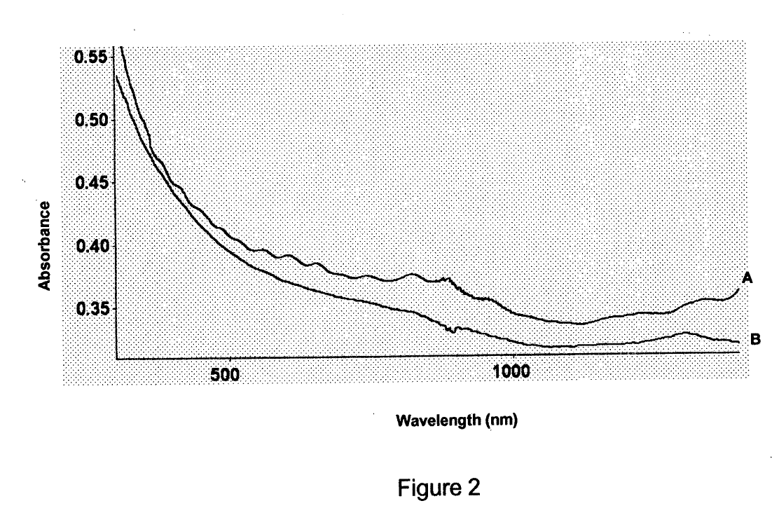

FIG. 3 shows FTIR spectra of the functionalized SWNTs obtained by using the attenuated total reflectance (ATR) attachment.

FIG. 4 shows thermal degradation analyses (TGA) of 1a, 1b, 2a, and 2b.

FIG. 5 shows a high resolution TEM image of 2b.

FIG. 6 shows a comparison of FTIR of the alkylated product (a-F-SWNT) to the starting material, F-SWNT.

FIG. 7 shows the XPS spectrum of a-F-SWNT.

FIG. 8 shows the Raman spectrum of a-F-SWNT.

FIG. 9 shows a SEM image of a fracture surface of a-F-SWNT 1% by weight in MDPE.

FIG. 10 shows Raman spectra of F-SWNT and U-F-SWNT.

FIG. 11. FTIR of spectra of F-SWNT and U-F-SWNT.

FIG. 12 shows TGA of U-F-SWNT made by the melt synthesis.

FIG. 13 an SEM image of U-F-SWNT made by the melt synthesis.

FIG. 14 an AFM of urea treated SWNT from liquid state.

FIG. 15 shows an AFM image of Urea treated F-SWNT in solid state.

FIG. 16 shows an AFM image converted into a height by color image by height conversion program.

FIG. 17 shows an AFM image of a urea treated nanotube with a specific concentration solution.

FIG. 18 shows TEM image of U-F-SWNT at scale of 20 nm.

FIG. 19 shows another TEM image of U-F-SWNT at scale of 20 nm.

FIG. 20 shows TEM image of U-F-SWNT at scale of 10 nm.

FIG. 21 shows another TEM image of U-F-SWNT at scale of 10 nm.

FIG. 22 shows the diameter distribution of urea treated nanotubes.

FIG. 23 shows a comparative bar graph of averaged strength from tensile test of the functionalized nanotubes in MDPE.

FIG. 24 shows FTIR spectra of derivitized F-SWNTs (U, G, and T) in comparison with the parent F-SWNT.

FIG. 25 shows Raman spectra of fluorinated (A) and derivatized nanotubes, U-F-SWNT (B), T-F-SWNT (C), and G-F-SWNT (D).

FIG. 26 shows XPS C1s and F1s spectra of functionalized SWNTs: F-SWNTs (A), U-F-SWNTs from urea melt synthesis (B) and from DMF solution synthesis (C), G-F-SWNTs (D) and T-F-SWNTs (E) both prepared at 100° C.

FIG. 27 shows TGA-DTA curves for (A) F-SWNTs, (B) Urea, (C) U-F-SWNTs produced by urea melt synthesis, (D) U-F-SWNTs from DMF solution synthesis, (E) T-F-SWNTs, (F) G-F-SWNTs.

FIG. 28 shows AFM images and height analysis for derivatized F-SWNT samples: (a) U-FSWNTs from DMF solution synthesis; (b) G-F-SWNTs; (c) T-F-SWNTs, height analysis across the nanotube; (d) T-F-SWNTs, height analysis along the nanotube backbone.

FIG. 29 shows a picture of F-SWNT and U-F-SWNT in water and 5% urea solution.

FIG. 30 shows a picture of F-SWNT and derivatives (U, G, T) in DMF.

FIG. 31 shows the Raman spectrum of APTES F-SWNT.

FIG. 32 shows TGA of APTES F-SWNT.

FIG. 33 shows the FTIR spectrum of APTES F-SWNT.

### DETAILED DESCRIPTION

In the following description, specific details are set forth such as specific quantities, sizes, etc. so as to provide a thorough understanding of embodiments of the present invention. However, it will be obvious to those skilled in the art that the present invention may be practiced without such specific details. In many cases, details concerning such considerations and the like have been omitted inasmuch as such details are not necessary to obtain a complete understanding of the present invention and are within the skills of persons of ordinary skill in the relevant art.

The present disclosure provides functionalized carbon nanotubes (CNTs) for incorporation into polymer composite materials. Without being bound by the mechanism, functionalized carbon nanotubes exhibit improved dispersion within polymer materials due to reduced bundling of the CNTs. Substituted CNTs may disrupt Van der Waals attraction between nanotubes allowing for better dispersion by conventional shear methods, for example. In alternate embodiments, the functionalized CNTs may be integrated covalently into a polymer backbone via functional group moieties present along the sidewalls and end caps of the CNTs.

Carbon nanotubes (CNTs), in accordance with embodiments of the present disclosure, include, but are not limited to, single-walled carbon nanotubes (SWNTs), multi-walled carbon nanotubes (MWNTs), double-walled carbon nanotubes (DWNTs), buckytubes, fullerene tubes, tubular fullerenes, graphite fibrils, and combinations thereof. Such CNTs can be made by any known technique including, but not limited to the HiPco RTM process, arc discharge, laser oven, flame synthesis, chemical vapor deposition (U.S. Pat. No. 5,374,415), wherein a supported or an unsupported metal catalyst may also be used, and combinations thereof. Depending on the embodiment, the CNTs can be subjected to one or more processing steps prior to subjecting them to any of the processes described in the present disclosure. In some embodiments, the CNTs have been purified. Exemplary purification techniques include, but are not limited to, those by Chiang et al. (Chiang et al., J. Phys. Chem. B 2001, 105, 1157; Chiang et al., J. Phys. Chem. B 2001, 105, 8297). The terms “CNT” and “nanotube” are used synonymously herein. Furthermore, while much of the discussion herein involves SWNTs, it should be understood that many of the methods and/or compositions of the present invention utilizing and/or comprising SWNTs can also utilize and/or comprise MWNTs or any of the other types of CNTs defined hereinabove.

In some embodiments, mixtures of various types of CNTs are employed, e.g., combinations of SWNTs and MWNTs. Such combinations of CNTs provide enhanced, synergistically-derived properties. Some CNTs can be initially supplied in the form of a fluff (felt), powder, pearls, and/or bucky paper. Alternatively the composite containing the alkyl-substituted carbon nanotube may be formed by mechanical dispersion of the nanotube within the polymer matrix. Such conventional processes may include, for example, extrusion which may additionally orient the CNTs within the polymer matrix.

SWNT dispersion in composite materials has been thwarted by the Van der Waals forces between CNTs, which cause the formation of large bundles. These bundles create unwanted effects such as decreasing the mechanical strength of polymer composites. Their provocative geometry, specifically their high aspect ratio of length to diameter, could provide materials with tensile strengths on the order of 60 GPa. Therefore, it would be beneficial to functionalize the SWNT sidewall in order to disrupt the π-π stacking interactions and Van der Waals forces between the SWNTs within the bundles, and thereby dramatically increase the availability of individual SWNTs. When SWNTs are in smaller bundles or as singles, dispersion may be improved in solutions and in composites which would enable many applications. These goals have been pursued by adopting various nanotube sidewall functionalization strategies through developing a number of covalent and non-covalent methods. The enhancement of properties of various application-based composites, coatings, and electronics, in particular, benefits from the covalent sidewall functionalization of SWNTs, which is capable of creating an efficient interface between the SWNTs and the matrix.

Fluorination of SWNTs was the first covalent sidewall functionalization method to produce the highly individualized and soluble nanotubes. Fluorination of CNTs alone has already resulted in increased dispersion in composites.

Fluorinated SWNTs can be further derivatized due to a higher reactivity than the pristine SWNTs. The fluorine in the C—F bond of F-SWNT can be readily substituted by a variety of nucleophilic reagents to produce an array of sidewall functionalized SWNTs. In particular, it was shown that the reactions of F-SWNT with terminal alkylidene diamines provide a convenient route to amino functionalized SWNTs through the sidewall C—N bond forming reactions. These reactions include the use of the other substituted amino compounds, such as aminoalcohols, aminothiols, aminoacids, and aminosilanes, for preparation of the SWNTs sidewall functionalized with the terminal OH, SH, COOH and silyl groups by the similar one-step route.

It should be noted that in comparison with the widespread approach to functionalization, which is based upon etching of nanotube surface by oxidative acids, the method of direct fluorination and subsequent substitution of fluorine generally causes no destruction to the SWNT sidewalls. This helps maintain the mechanical strength of the SWNT frame.

One exemplary polymer composite, in accordance with the present disclosure, includes a polymer matrix into which an alkyl-substituted carbon nanotube (a-SWNT) has been incorporated. The term “alkyl”, alone or in combination, means an acyclic alkyl radical, linear or branched, preferably containing from 1 to about 20 carbon atoms, for example, and such as 6 to about 12 carbon atoms, in another embodiment. The alkyl radicals can be optionally substituted as defined below. Examples of such radicals include methyl, ethyl, chloroethyl, hydroxyethyl, n-propyl, isopropyl, n-butyl, cyanobutyl, isobutyl, sec-butyl, tert-butyl, n-pentyl, amino-n-pentyl, iso-amyl, hexyl, octyl, decyl, undecyl, dodecyl and the like.

The term “optionally substituted” means the alkyl group may be substituted or unsubstituted. When substituted, the substituents may include, without limitation, one or more substituents independently chosen from: (C₂-C₈)alkenyl, (C₂-C₈)alkynyl, (C₁-C₈)heteroalkyl, C₈)haloalkyl, (C₂-C₈)haloalkenyl, (C₂-C₈)haloalkynyl, (C₃-C₈)cycloalkyl, phenyl, (C₁-C₈)alkoxy, phenoxy, (C₁-C₈)haloalkoxy, NH₂, (C₁-C₈)alkylamino, (C₁-C₈)alkylthio, phenyl-S—, oxo, (C₁-C₈)carboxyester, (C₁-C₈)carboxamido, (C₁-C₈)acyloxy, H, halogen, CN, NO₂, NH₂, N₃, NHCH₃, N(CH₃)₂, SH, SCH₃, OH, OCH₃, OCF₃, CH₃, CF₃, C(O)CH₃, CO₂CH₃, CO₂H, C(O)NH₂, pyridinyl, thiophene, furanyl, (C₁-C₈)carbamate, and (C₁-C₈)urea. Two substituents may be joined together to form a fused five-, six-, or seven-membered carbocyclic or heterocyclic ring consisting of zero to three heteroatoms. An optionally substituted group may be unsubstituted (e.g., —CH₂CH₃), fully substituted (e.g., —CF₂CF₃), monosubstituted (e.g., —CH₂CH₂F) or substituted at a level anywhere in-between fully substituted and monosubstituted (e.g., —CH₂CF₃).

The polymer matrix of the composite may include, without limitation, thermoset and thermoplastic materials. Examples of thermosets include, but are not limited to phenol formaldehyde resins, epoxy resins, melamine resins, vulcanized rubber, and polyester resins. Thermoplastics may include, but are not limited to, acrylonitrile butadiene styrene (ABS), celluloid, cellulose acetate, ethylene-vinyl acetate (EVA), ethylene vinyl alcohol (EVAL), fluorinated ethylene-propylene (FEP), perfluoroalkoxy polymer resin (PFA), chlorotrifluoroethylene (CTFE), ethylene chlorotrifluoroethlyene (ECTFE), ethylene tetrafluoroethylene (ETFE), polyacetal (POM), polyacrylates, polyacrylonitrile (PAN), polyamide (PA), polyamide-imide (PAI), polyaryletherketone (PAEK), polybutadiene (PBD), polybutylene (PB), polybutylene terephthalate (PBT), polyethylene terephthalate (PET), polycyclohexylene dimethylene terephthalate (PCT), polycarbonate (PC), polyhydroxyalkanoates (PHAs), polyketone (PK), polyester, polyethylene (PE), polyetheretherketone (PEEK), polyetherimide (PEI), polyethersulfone (PES, polyethylenechlorinates (PEC), polyimide (PI), polylactic acid (PLA), polymethylpentene (PMP), polyphenylene oxide (PPO), polyphenylene sulfide (PPS), polyphthalamide (PPA), polypropylene (PP), polystyrene (PS), polysulfone (PSU), polyvinyl chloride (PVC), and polyvinylidene chloride (PVDC).

Generation of alkyl-substituted carbon nanotubes: Examples of sidewall derivatization chemistry of SWNTs are still fairly limited. Useful functionalization methods include radical additions involving perfluoroalkyl and aryl radicals, produced photochemically or by electrochemical reduction, in contrast to a larger variety of known radical reactions of fullerenes.

Applicants have reported the functionalization of SWNTs by covalent sidewall attachment of free radicals thermally generated from organic peroxides, such as lauroyl and benzoyl peroxides [Peng, H.; Reverdy, P.; Khabashesku, V. N.; Margrave, J. L. Chem. Comm. 2003, 362-363], commonly used as radical initiators in polymerization reactions. Commercial availability of these peroxides as well as the ESR data showing the addition to C₆₀ of radicals, produced by photolysis or thermolysis of some peroxides, have facilitated characterization. Besides pristine SWNTs the same reactions may be carried out on fluorinated derivatives (F-SWNTs) as shown in Scheme 1b below. Both the solid-state and the solution phase reactions have been demonstrated.

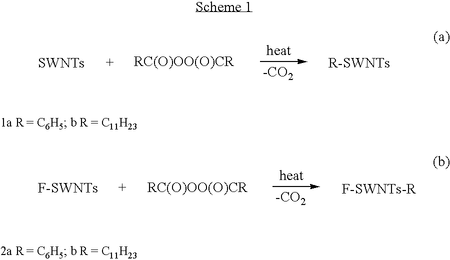

Raw SWNTs can be prepared by the HiPco process and can be thoroughly purified before further use to remove iron impurities. F-SWNTs can be prepared by direct fluorination of purified SWNTs to approximately C₂F stoichiometry according to literature procedures [Gu, Z.; Peng, H.; Hauge, R. H.; Smalley, R. E.; Margrave, J. L. Nano Lett. 2002, 2:1009]. Benzoyl peroxide was purchased from Fluka and lauroyl peroxide from Aldrich.

Example procedures: In example reactions presented herein about a 1 to 2 weight ratio of SWNTs material to peroxide was used, although other weight ratios may be used. In the solid state reactions a mechanically ground mixture of reactants was placed into a stainless steel reactor which was sealed and then heated at 200° C. for 12 h. The solution phase reactions were carried out by dispersing the SWNTs samples in o-dichlorobenzene by ultrasonication, adding the corresponding peroxide and refluxing the mixture under nitrogen at 80-100° C. for 3-120 h thereafter. The functionalized SWNTs 1a-b and 2a-b were isolated by washing off the unreacted peroxides and by-products with a large amount of chloroform on 0.2 μm pore size Teflon filter; the produced black film was peeled off and then dried in a vacuum oven at 100° C. overnight. The characterization of functionalized SWNTs 1a-b, 2a-b was performed by Raman, FTIR, and UV-Vis-NIR spectroscopy as well as TGA/MS, TGA/FTIR, and TEM data as described below.

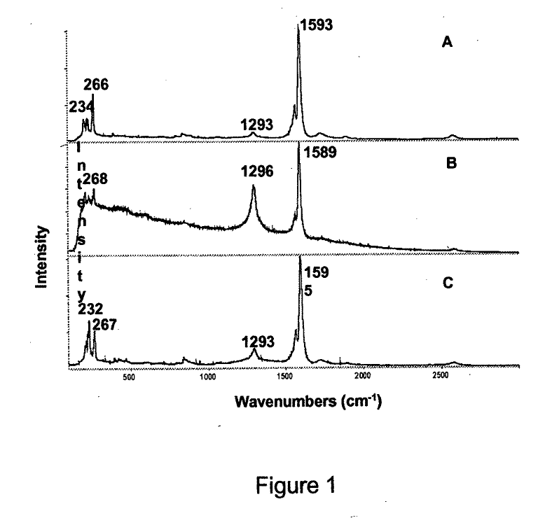

Raman and UV-Vis-NIR spectra in FIGS. 1 (showing Raman spectra of pristine SWNTs, trace A, phenylated SWNTs 1a, trace B, and phenylated SWNTs 1a after TGA, trace C) and 2 (showing comparison between unfunctionalized SWNTs, trace A, and phenylated SWNTS 1a, trace B), respectively, showed clear evidence for the significant alteration of the electronic states of 1a-b and 2a-b. In the Raman spectra, the observed decrease of the typical purified HiPco-SWNTs breathing and tangential mode peaks at 200-263 and 1591 cm⁻¹, respectively, along with the substantial increase of the sp³ carbon peak at 1291 cm⁻¹ provide a diagnostic indication of disruption of the graphene π-bonded electronic structure of the side walls, suggesting their covalent functionalization. This is further confirmed by their solution-phase UV-Vis-NIR spectra which show the diagnostic complete loss of the van Hove absorption band structures, routinely observed in purified HiPco-SWNTs.

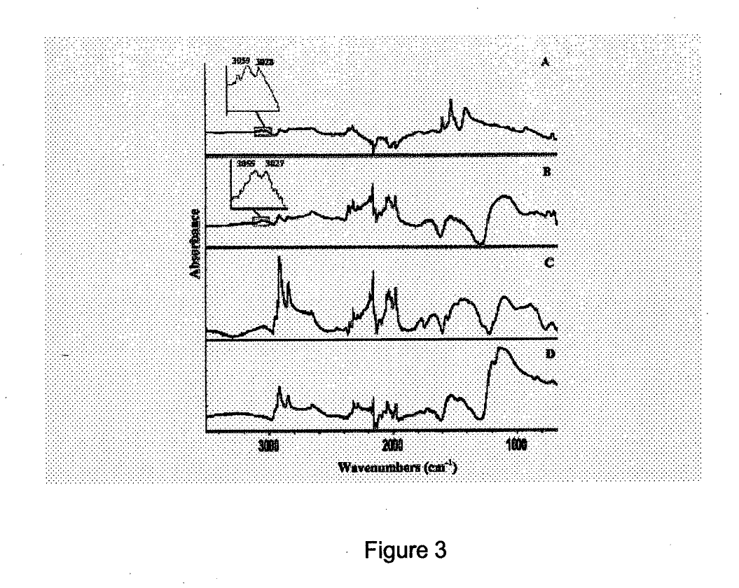

The FTIR spectra of the functionalized SWNTs, obtained by using the attenuated total reflectance (ATR) attachment, are shown on FIG. 3. The weak peaks in the 3060-3020 cm⁻¹ range in the spectra of 1a, b (shown in FIG. 3 under A and B, respectively) characterize the aromatic C—H stretches of phenyl groups attached to the SWNTs, while the peaks at 2919 and 2850 cm⁻¹, which appear after washing the reaction product with CHCl₃ followed by drying in a vacuum oven, belong to aliphatic C—H stretches. Several stronger absorptions in the 1600-1400 cm⁻¹ can be attributed to phenyl ring stretches and a broad peak at 1105 cm⁻¹ to the C—F stretch in 1b. The attachment of long chain undecyl groups to SWNTs and F-SWNTs is indicated in the spectra of 2a,b (shown on FIG. 3 under C and D, respectively) by observation of prominent peaks of the C—H stretches in the 2980-2800 cm⁻¹ range and an absorption of C—H deformation mode at 1465 cm⁻¹. In addition, a mid-intensity band at 1547 cm⁻¹ and a doublet at 1202, 1145 cm⁻¹ due to an activated C═C and a residual C—F stretches, respectively, are present in the spectra of 2b. Based on the relative intensities of the C—H stretching modes in IR spectra of products 1, 2, it is reasonable to suggest that the radical additions to the F-SWNTs proceed more readily which is in line with their stronger than pristine SWNTs electron accepting ability. This is also indicated by a much shorter reaction time (3 h vs. 5 days) required in case of F-SWNTs for observation of prominent C—H peaks in the FTIR spectra of nanotube derivative 2b in comparison with the SWNT-derived product 2a.

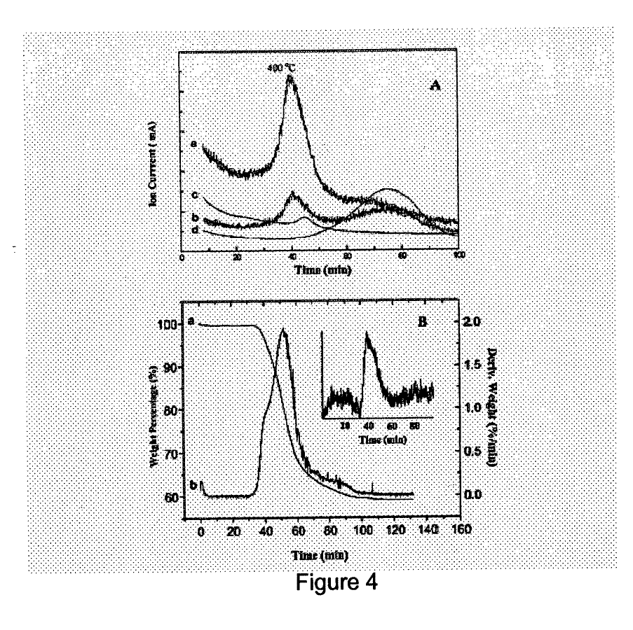

Further evidence for covalent functionalization of SWNTs has been provided by thermal degradation analyses (TGA) of 1, 2 in the 50-1000° C. range coupled with the on-line monitoring of volatile products either by MS or FTIR techniques. For instance, the TGA/MS data of 1a in FIG. 4a show the evolution of detaching phenyl radicals at 400° C., indicated by a major peak on the m/z 77 and a smaller peak on the m/z 78 ion current vs. time plots (a) and (b), respectively, and their partial dimerization to biphenyl (m/z 154) volatizing at a higher temperature (plot (d)). These data confirm that the detected phenyl radicals originate from the functionalized SWNTs and not from the reaction by-products, such as biphenyl or benzoic acid ester C₆H₅COOC₆H₅, indicated by a very small peak on the m/z 105 plot (c). The TGA/FTIR analysis of another sample, 2b in FIG. 4b, also shows on a derivative plot (b) a major peak at about 400° C. which corresponds to the loss of undecyl radicals by 2b. This was confirmed by synchronizing this peak with the maximum on the chemigram of the C—H stretch region (2800-2980 cm⁻¹) in FTIR spectra of volatile products (inset on FIG. 4b). Analysis of the same SWNT-derivative, 2b, by variable temperature pyrolysis-EIMS confirmed the TGA/FTIR data by indicating the major loss of undecyl radical, C₁₁H₂₃, and their dimer at about 350-400° C. (peaks in ELMS at m/z 155 and 310, respectively). It is important to note, that the thermal degradation of functionalized SWNTs results in formation of bare wall nanotubes, indicated by restoration of their features in the Raman spectra taken for solid residues after TGA analyses.

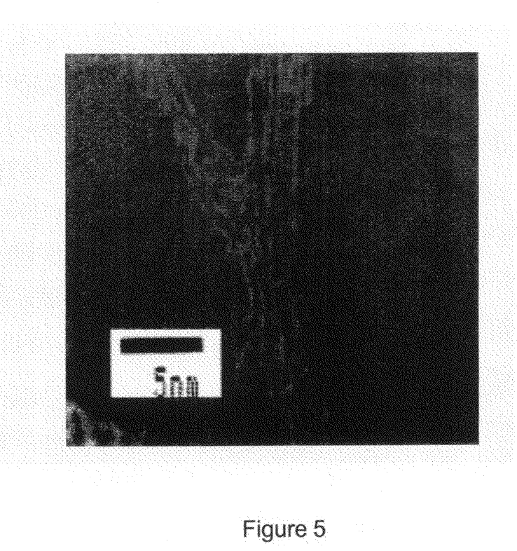

The covalent attachment of a bulky long-chain group, such as undecyl, provided an opportunity to directly observe the functionalized SWNTs by TEM. Indeed, a high resolution TEM image of 2b specimen in FIG. 5 clearly shows individual nanotubes with long-chain substituents joined to their sidewalls.

The reactions of benzoyl peroxide with the SWNTs and F-SWNTs were found to proceed more readily in the solid state, while functionalization using lauroyl peroxide has been found more efficacious in the solution phase. It was also observed that the same reactions proceed much faster with C₆₀ as the substrate which reacts in only a few hours. By comparison, pristine SWNTs, having significantly lower sidewall curvature, require several days. Besides using the free radicals produced by the thermal decomposition of acyl peroxides to functionalize the SWNTs, it is expected that functionalization may be achieved using other organic peroxides and radical precursors known in the art, such as alkyl halides, alkyl tins and the like. Additionally, other carbon nanostructures, e.g., multi-walled carbon nanotubes, fullerenes, polyfullerenes, and graphite may serve as a substrate for functionalization.

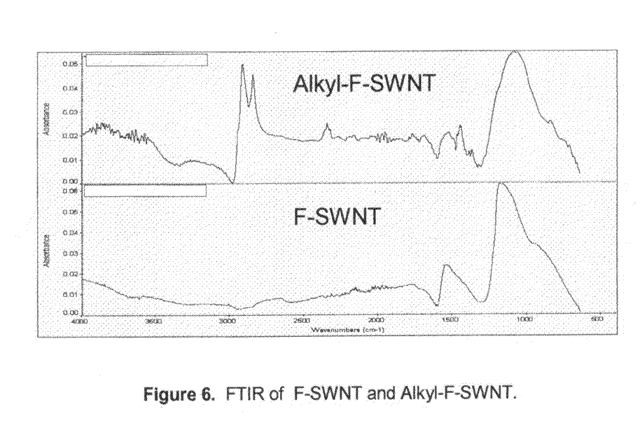

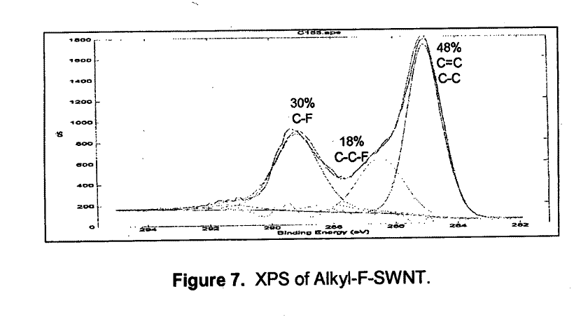

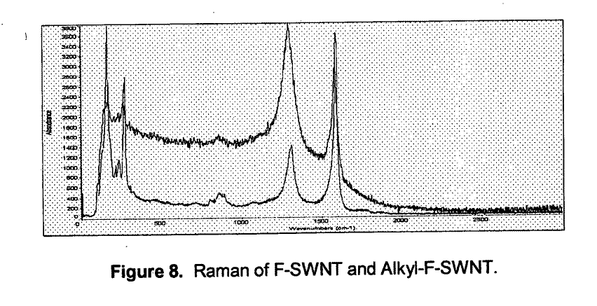

Incorporation into polymer composite: Alkyl-substituted carbon nanotubes may be incorporated into the polymer composite by conventional mechanical means as shown in this following exemplary embodiment. Lauroyl peroxide was used as described above to modify fluorinated single walled carbon nanotubes from Carbon Nanotechnologies Inc. (CNI). The characterization of these alkylated fluorinated carbon nanotubes (a-F-SWNT) is shown in FIGS. 6-8 (Please confirm these are the lauroyl data). FIG. 6 shows a comparison of FTIR of the alkylated product (a-F-SWNT) to the starting material, F-SWNT. FIG. 7 shows the XPS spectrum confirming addition of the alkyl group and FIG. 8 shows the Raman spectrum of a-F-SWNT.

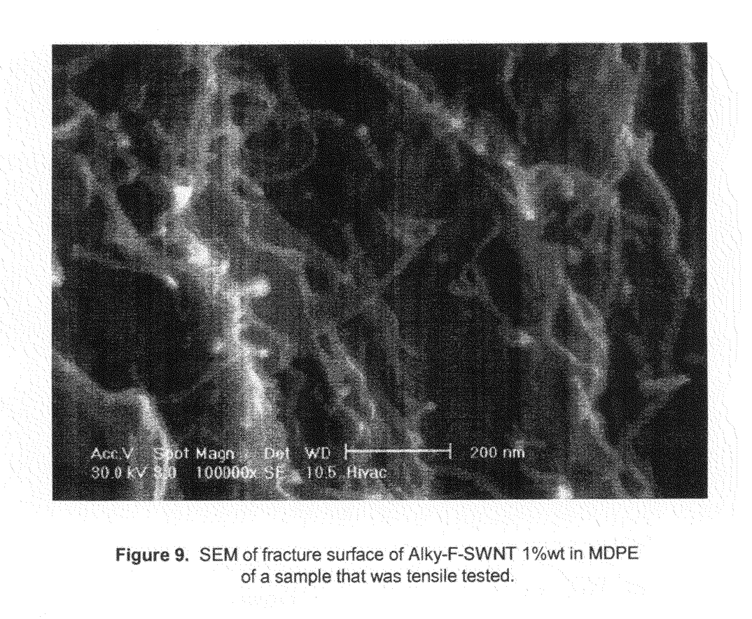

a-F-SWNTs were incorporated into a polymer matrix by the following example procedure: (1) Sonicating 0.2 g long chain alkyl [—C₁₁H₂₃] fluorinated functionalized nanotubes (a-F-SWNTs) in 250 ml chloroform for 30 minutes to form solvent-dispersed nanotubes; (2) Rotary evaporating the solvent-dispersed nanotubes and 19.8 g of medium density polyethylene (MDPE) powder to form an overcoated mixture; and (3) Shear mixing the overcoated mixture for 15 minutes and heat/pressure molding it into thin panels from which dogbone-shaped samples were cut out for tensile testing. FIG. 9 shows an scanning electron microscope (SEM) image of the product composite, having about 1% by weight a-F-SWNT. Further data concerning the properties of this composite are discussed hereinbelow.

After shear mixing, the composite material may be further processed by passing through an extruder, for example, which may serve to orient the functionalized carbon nanotubes within the polymer matrix. This may enhance, for example, electrical conductive properties of the composite. It should be appreciated that the raw composite may be subjected to other procedures known in the art, such as deposition modeling, and fiber spinning, which includes, but is not limited to melt spinning, wet spinning, dry spinning, and gel spinning, for example.

In alternate embodiments, a functionalized SWNT bearing a functional group may be incorporated into a polymer matrix by forming covalent links within the matrix. This may be carried out during polymerization. For example, a-SWNTs or a-FSWNTs displaying terminal alkenes may be readily incorporated into a polystyrene polymer matrix by mixing the a-SWNT or a-F-SWNT with styrene and then performing the polymerization by conventional means, such as radical polymerization. In other embodiments, the a-SWNTs may be covalently linked to an already established polymer backbone by conventional synthetic methods. For example, an a-SWNT or a-F-SWNT displaying a carboxylic acid functional group may be tied covalently into a polyvinyl alcohol (PVA) backbone through routine esterification chemistry.

The present disclosure also contemplates a polymer composite that includes incorporating a fluorinated carbon nanotube that has been functionalized with a compound of formula I into the polymer matrix:

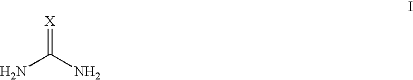

X may be O (urea, U), S (thiourea, T), and NH (guanidine, G). Again the polymer matrix may be a thermoset or thermoplastic material as described above. The fluorinated carbon nanotube functionalized with urea, thiourea, or guanidine may form a covalent link within the polymer matrix via a pendant NH₂ group. These compounds were chosen due to their low cost, water solubility and chemical properties prompting their use as chemical synthons for production of plastics, resins, rubber chemicals, rocket propellants and biomaterials. Urea, thiourea and guanidine are also chaotropic agents which can cause disruption of local non-covalent bonding in molecular structures, particularly, hydrogen bonding in water. This interaction has been studied in protein solutions [Israelavachvili, J. Intermolecular and Surface Forces. 2nd Ed. Elsevier Academic Press. 1992. p. 135; Nemethy, G. Angew. Chem. Int. Ed. 1967, 6:195] and more recently with SWNTs [Ford, W. E.; Jung, A.; Hirsch, A.; Graupner, R.; Scholz, F.; Yasuda, A., Wessels, J. M. Adv. Mater. 2006, 18:1193-1197]. Since F-SWNTs are hydrophobic, urea can intercalate nanotube bundles by disrupting the Van der Waals forces, and self-assemble around SWNTs until unbundling occurs. Similar behavior is commonly noted in urea-based protein folding solutions [Israelavachvili et al.]. For these reasons, the covalent attachment of simple amide and heteroamide moieties to the SWNT sidewalls is expected to result in smaller SWNT bundles and improved dispersion in water and polar organic solvents.

Access to, fluorinated carbon nanotubes bearing U, T, or G may be obtained by heating a fluorinated carbon nanotube with the parent compound of formula I:

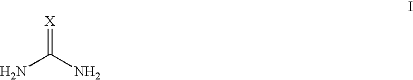

The suggested reactions are shown on schemes 2 and 3 below.

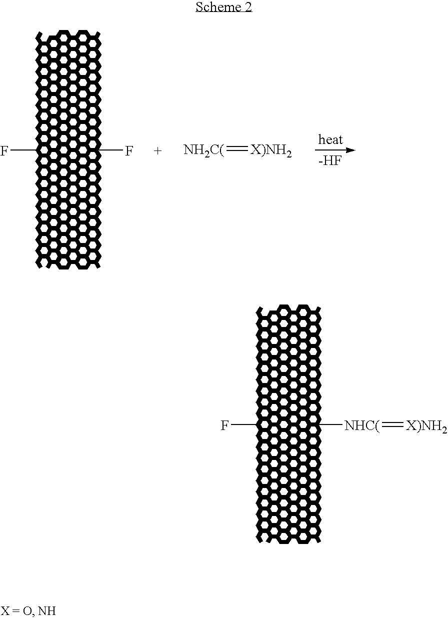

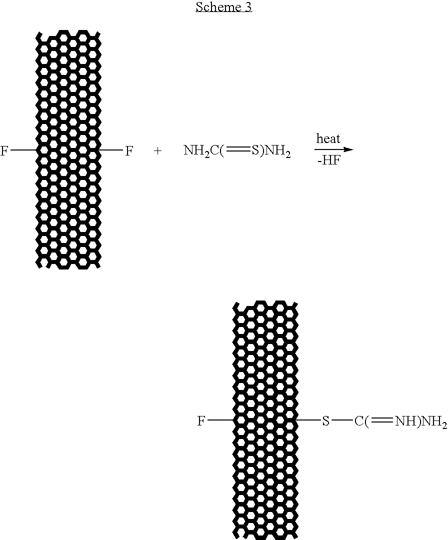

Unlike urea and guanidine, which react with the F-SWNTs through their NH₂ groups and form C—N linkages with the SWNT sidewalls after elimination of HF (Scheme 2), thiourea most likely attaches to the sidewall not through the C—N but the C—S bond (Scheme 3). This is deemed possible in view of higher nucleophilicity of sulfur in the >C═S moiety relatively to oxygen in the >C═O and nitrogen in the >C═NH groups [Speziale, A. J. Org. Synth., Coll., 1963, 4:401; The chemistry of double-bonded functional groups, Ed. S. Patai, John Wiley and Sons. New York, N.Y., 1977, pp. 1355-1496]. The relative weakness of the C═S double bond compared to C═N and C═O double bonds has been attributed to poor orbital matching between the relatively large sulfur atom and the smaller carbon atom. Thus, where compounds containing C═S double bond exhibit potential ambident nucleophilicity reaction through sulfur is generally thermodynamically favored.

Under prolonged heating, up to its melting point, urea can undergo polymerization as well as decomposition with release of ammonia and formation of isocyanic acid. Therefore, these processes are expected to contribute to the functionalization reaction of F-SWNTs with urea and result in attachment of some polyurea (PolyU) units as well to the sidewalls of F-SWNTs to form PolyU-F-SWNT derivatives according to the following equations:

F-SWNT-NHCONH₂+nH₂NCONH₂→NH₃+F-SWNT-NHCONH(CONH)ₙH (1)

PolyU-F-SWNT

H₂NCONH₂ [symbol] NH₃+HNCO (2)

F-SWNT-NHCONH₂+nHNCO [symbol] F-SWNT-NHCONH(CONH)ₙH (3)

PolyU-F-SWNT

These secondary processes most likely occur to different degrees during the urea melt and solution synthesis conditions employed in the present work. The addition reactions of isocyanic acid in molten urea are reversible according to the recently proposed mechanism for the reaction of oxidized SWNTs with urea melt where formation of some polyurea-derivatized nanotubes was observed. Under heating and stirring of urea and F-SWNTs in DMF solution in the presence of pyridine for 4 hours at 100° C., the formation of polyurea can become more noticeable. Other secondary reactions can also occur, particularly hydrolysis of urea moieties in the U-F-SWNTs to produce carbamic acid groups —NHC(═O)OH on the SWNT sidewalls as reactive intermediates. The latter can react with isocyanic acid, and thus, serve as building blocks for incorporation of urethane units into a PolyU-F-SWNT side chain. In comparison, formation of the polymerization by-products stemming from the SWNT sidewalls during the functionalization of F-SWNTs with thiourea and guanidine hydrochloride under similar DMF solution synthesis conditions is not as likely.

The reactions of F-SWNTs shown on Schemes 2 and 3 also produce gaseous HF as a by-product which under urea melt process conditions will most likely completely evaporate while during the solution process HF can dissolve in DMF and form ammonium-type salts by bonding to certain heteroamide functional groups attached to the SWNTs. Salt formation may be more evident in the case of guanidine-SWNTs (G-F-SWNTs) due to greater basicity of guanidine as compared to thiourea and urea.

The following procedure for functionalizing fluorinated (and nonfluorinated) tubes, F-SWNTs serves as an example: (1) Melt 2 g of urea crystals and mix with 20 mg of fluorinated nanotubes under nitrogen for four hours to form a mixture. (2) Cool and wash the cooled mixture with purified water in a sonic bath for 20 minutes. (3) Filter the washed mixture with a PTFE membrane and dry the collected product (urea fluorinated nanotubes, U-F-SWNT) in a vacuum oven.

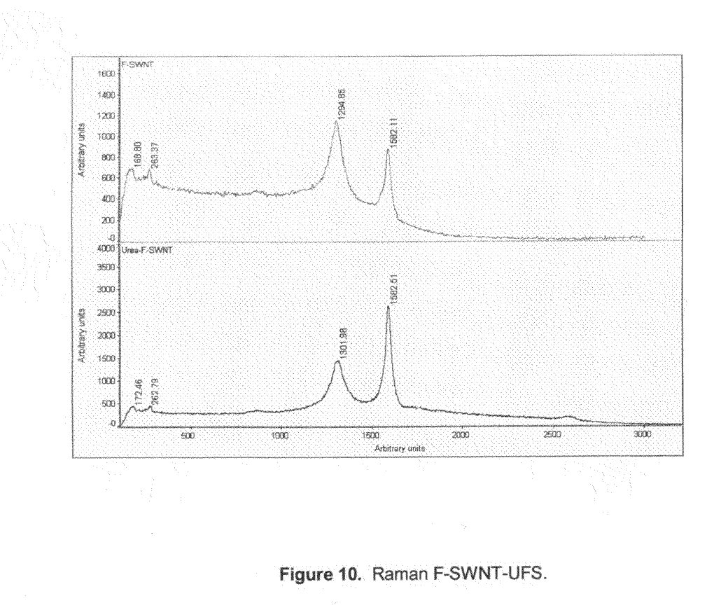

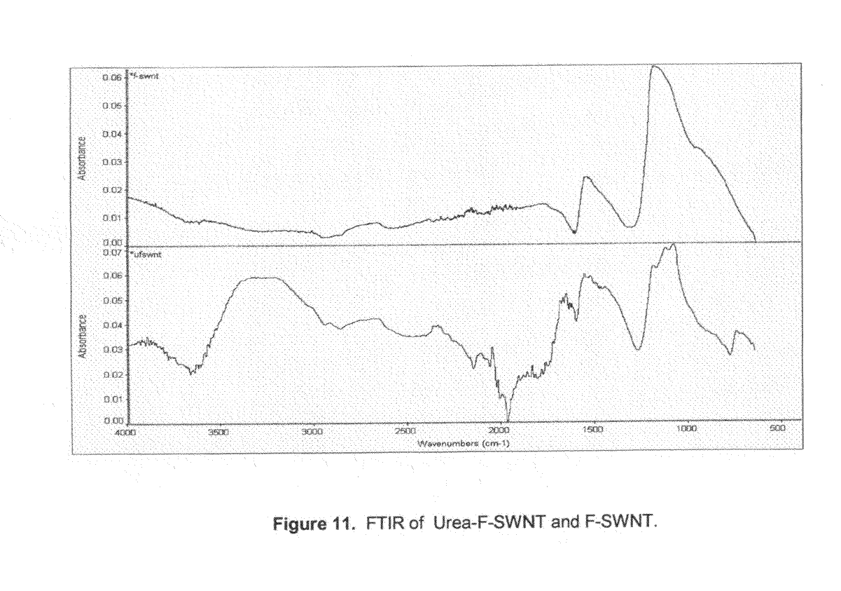

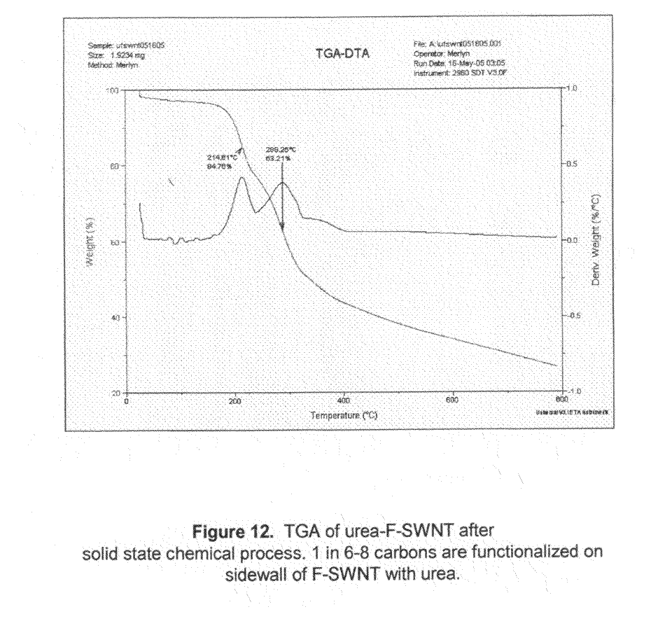

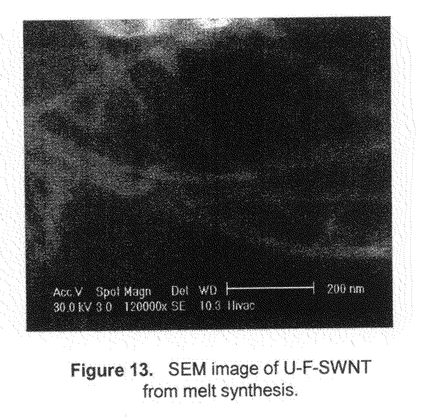

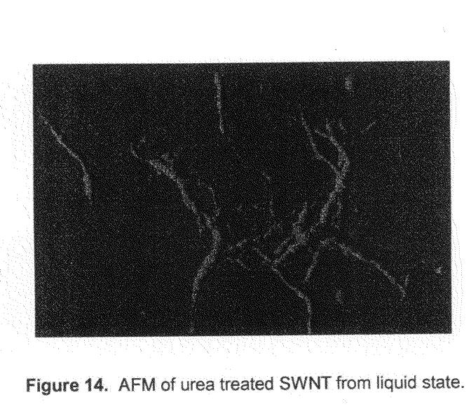

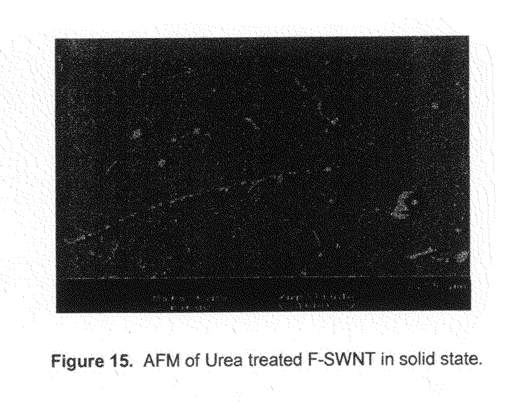

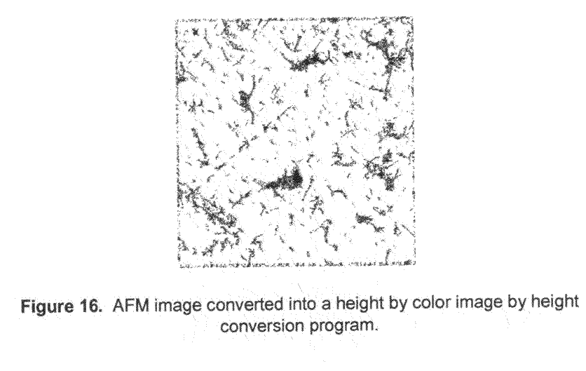

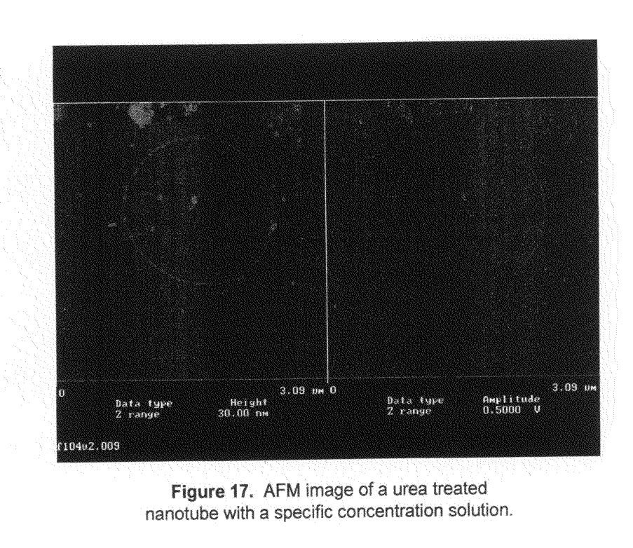

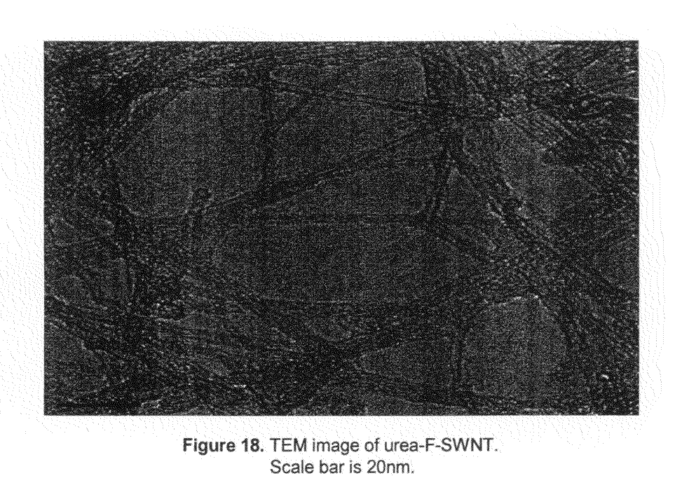

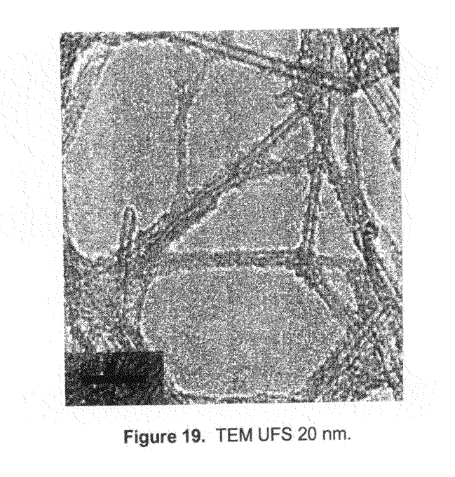

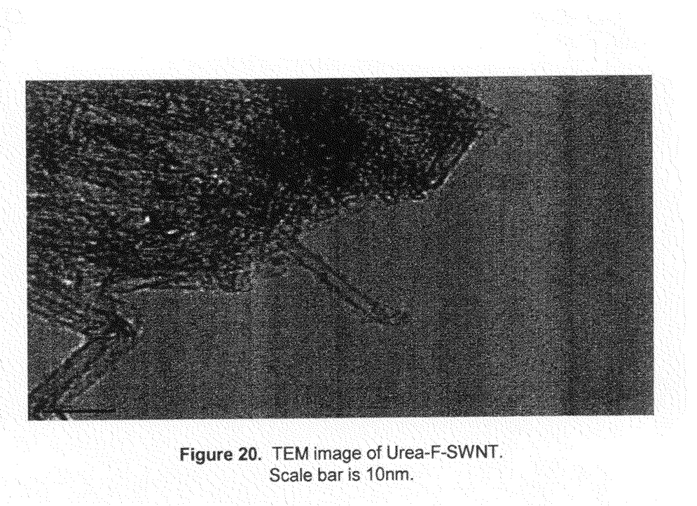

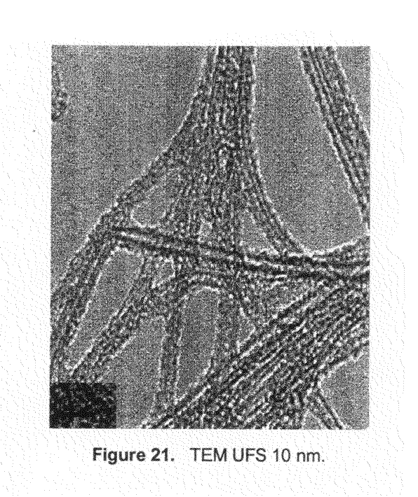

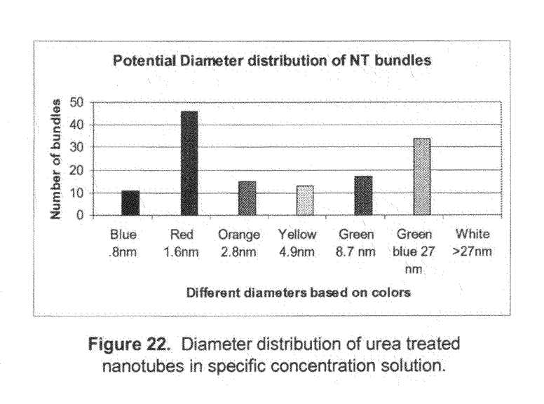

Once synthesized, the urea fluorinated nanotubes (U-F-SWNTs) can then be incorporated into the MDPE the same way as the a-F-SWNT described herein above. FIGS. 10-22 show extensive characterization of U-F-SWNTs. FIG. 10 shows a side by side comparision of the Raman spectra for F-SWNT and U-F-SWNT. A similar comparison of FTIR spectra is shown in FIG. 11. FIG. 12 shows the thermogravimetric analysis of U-F-SWNT made by the melt process. Based on the TGA plot, about 1 in every 6-8 carbons are functionalized on the sidewalls of the F-SWNT starting material. FIG. 13 shows an SEM image of U-F-SWNT from the melt synthesis. FIGS. 14-17 show various AFM images of urea treated F-SWNTs, both from the melt synthesis and solution synthesis. Similarly, TEM images of U-F-SWNTs at different scales are shown in FIGS. 18-21. FIG. 22 shows the distribution of bundles according to size for urea treated F-SWNTs.

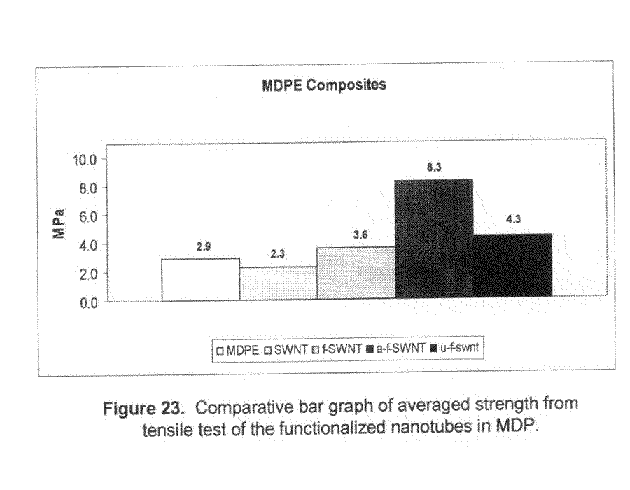

As shown in FIG. 23, the tensile test of 1% a-F-SWNT/MDPE composites show a dramatic increase of 185% over neat MDPE, although the tensile test showed only a 48% increase for MDPE composites loaded with 0.5% wt urea-F-SWNTs. It is believed that the addition of the urea-F-SWNT into epoxy would more significantly increase mechanical properties of epoxy composites due to the amide terminated ends of urea.

Experimental Details Materials: Urea with 99% purity was purchased from Sigma-Aldrich Chemical Company, Inc. (Milwaukee, Wis.). Guanidine in the form of guanidinium hydrochloride (98% pure) was acquired from Alfa Aesar (Ward Hill, Mass.). Thiourea was purchased from Sigma-Aldrich Chemical Company, Inc. (Milwaukee, Wis.). F-SWNTs of approximately C₂F stoichiometry were obtained from Carbon Nanotechnologies, Inc. (Houston, Tex.).

Methods: Urea-functionalized SWNTs (U-F-SWNTs) were prepared from F-SWNTs by using two methods, solvent-free urea melt synthesis, and solution synthesis. In an exemplary urea melt synthesis, 50 mg of F-SWNTs were mixed with 5 g of urea and ground in a mortar. The mixture was placed into a three-neck flask, heated to 150° C. to melt and stirred at this temperature for 4 hours under nitrogen. Thereafter, the mixture was cooled to room temperature, de-ionized water was added into the flask and the mixture sonicated for 30 minutes in a bath sonicator. The solution was then filtered on a Millipore Fluoropore PTFE filter membrane with a 0.22 μm pore size. The product was washed repeatedly with de-ionized water and ethanol and then dried overnight in a vacuum oven at 70° C. In the solution synthesis method, 50 mg of F-SWNTs were sonicated in DMF for 20 minutes and 500 mg of urea added afterwards with 10 drops of pyridine. The mixture was heated and stirred at 100° C. under nitrogen for 4 hours. The product was collected on a filter membrane after washing off unreacted urea with de-ionized water and ethanol.

Solution synthesis method was also applied for preparation of thiourea-functionalized SWNTs (T-F-SWNT) according to the following example: 50 mg F-SWNT was sonicated in 100 ml DMF, followed by addition of 500 mg of thiourea, and ten drops of pyridine. The solution mixture was then heated and stirred at 80° C.-100° C. under nitrogen for 4-12 hours. Higher temperature conditions were not desirable since thiourea decomposes above 135° C. The mixture was cooled down to room temperature and washed repeatedly with de-ionized water and ethanol, and dried overnight in a vacuum oven at 70° C. The guanidine-functionalized SWNTs (G-F-SWNT) derivative was prepared by sonicating 50 mg of F-SWNTs with DMF for 20 minutes, then 500 mg of guanidine hydrochloride and ten drops of pyridine were added to the solution. The mixture was heated to 100° C. and stirred under nitrogen for 4 hours. Afterwards, the SWNT were similarly washed and dried overnight in a heated vacuum oven.

Characterization. F-SWNTs and the synthesized U-F-SWNT, T-F-SWNT, and G-F-SWNT derivatives were characterized by the Raman, FTIR, XPS, TGA, SEM/EDX, and TEM methods. For Raman spectroscopy, a Renishaw Microraman system operating with a 780 nm AlGaAs diode laser source was used. ATR-FTIR spectral measurements were performed using a Thermo Nicolet Nexus 670 FTIR spectrometer on samples pressed into a KBr pellets. Thermal degradation analyses (TGA) were done in inert environment using pre-purified argon gas with a TA-SDT-2960 TGA-DTA analyzer. X-ray photoelectron spectroscopy (XPS) data for elemental analysis were obtained with PHI Quantera spectrometer using the monochromatic Al Kα radiation source (1486.6 eV) with a power setting of 350 W and an analyzer pass energy of 23.5 eV. For atomic force microscopy (AFM) analysis a Digital Instrument Nanoscope IIIA with Silicon tip was used. Transmission electron microscopy (TEM) images of specimen placed on lacey carbon coated copper grids (size 300 mesh) were obtained with a JEOL JEM-2010 electron microscope operating at an accelerating voltage of 100 kV for microstructure investigation. Environmental thermal field emission electron microscope (SEM) FEI XL-30 with 2 nm high resolution was used for surface imaging.

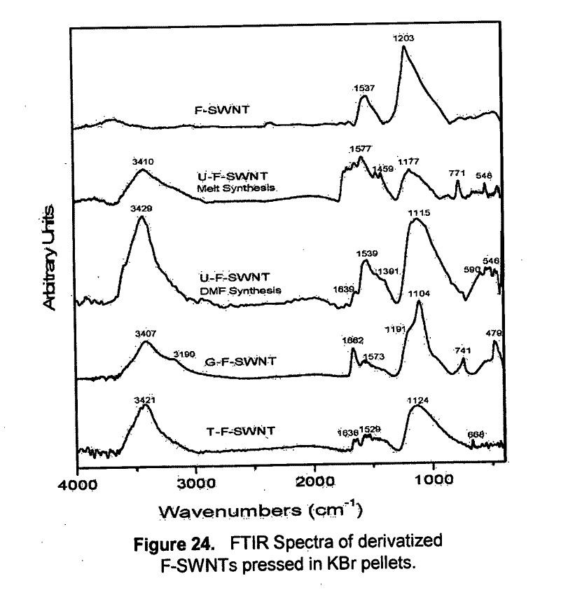

FTIR spectroscopy. The FTIR spectra of functionalized SWNTs are shown on FIG. 24. They provide structural information on the functional groups present on the SWNT sidewall before and after the reaction. In the spectrum of F-SWNT sample the absorption band of the C—F stretch shows at 1203 cm⁻¹, while the band of activated sidewall C═C stretches is detected near 1537 cm⁻¹ in agreement with the IR characterization data on fluorinated HipCO SWNTs. In the spectra of derivatized nanotubes, such as U-F-SWNTs, prepared both by melt and solution syntheses, G-F-SWNTs, and T-F-SWNTs, strong absorption bands at 3400-3430 cm⁻¹ attributed to the N—H stretches, are seen. Peaks observed in these spectra in the 1700-1500 cm⁻¹ range characterize the C═O and C═N stretches coupled with the in-plane NH and NH₂ bending vibrations of the (C═O)NH moieties in U-F-SWNTs and (C═NH)NH₂ units in G-F-SWNTs and T-F-SWNTs.

Medium intensity or shoulder bands appearing in these spectra in the 1500-1350 cm⁻¹ region are due to antisymmetric C—N stretching vibrations coupled with the out-of-plane NH₂ and NH modes. The band of the stretching mode of residual sidewall C—F groups is significantly weakened in the spectra of derivatized F-SWNTs because of partial removal of fluorine and appears as a shoulder on a broad band in the 1200-950 cm⁻¹ region. Besides C—F stretching mode, out-of-plane NH and NH₂ and symmetric C—N stretching vibrations also contribute into an observed high-intensity of this band. The band at 771 cm⁻¹ in the IR spectrum of urea is normally assigned to the CO deformation mode coupled with the antisymmetrical NH₂ torsional mode. Therefore, we have assigned the peak appearing in the similar position in the spectra of U-F-SWNTs to this type of vibration. By comparison with literature data on guanidinium salts, the peak at 741 cm⁻¹ in the spectrum of G-F-SWNTs is assigned to the out-of-plane NCNN deformation mode. In the IR spectra of free thiourea the peak at 730-740 cm⁻¹ is assigned to the C═S stretching vibration. It was found that this mode shifts to lower wavenumber by about 20-25 cm⁻¹ in the coordination compounds of thiourea with ZnSO₄ and CdCl₂ due to somewhat weakened C═S bond. For this reason, it is expected that when thiourea bonds covalently to the F-SWNT sidewall through nucleophilic sulfur, the frequency of the C—S single bond stretch in the T-F-SWNTs will downshift further. This argument supports the assignment of the observed peak at 668 cm⁻¹ in the spectrum of T-F-SWNTs to this mode.

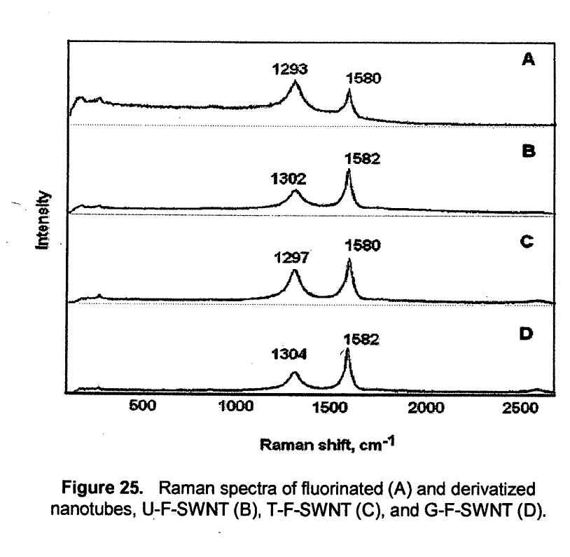

Raman spectroscopy. The Raman spectra of the F-SWNTs (FIG. 25A) and derivatized products (FIG. 25B-D) show a decreased intensity and shift of the disorder peak (D mode) as compared to the F-SWNTs. The decreased intensity of the D peak is due to the reduction in the number of sp³ C—C bonded carbons, as some of the fluorine atoms are removed from the sidewall and substituted through the predominant sidewall-C—X (X═N or S) covalent bond formation according to the reaction schemes (Schemes 2, 3). Attaching the urea and heteroamide moieties also results in some recovery of sp² bonds between the sites of sp³ C—X attachment and partial restoration of aromatic π-electron conjugation and graphene symmetry along the length sections of the sidewall which is reflected by increase in the intensity of G-peak at 1580-1582 cm⁻¹ (FIG. 25B-D).

The observed high intensity of D-peak in the Raman spectrum of F-SWNTs (FIG. 25A) reflects the largest content of the sp^{a}-hybridized sidewall carbons. (˜37 at. %) among all functionalized SWNTs prepared in the present work. The shift in the position of D peak in the spectra of derivatized F-SWNTs indicates that instead of fluorine another group is covalently attached to the sidewall. Of all the Raman spectra, the G-F-SWNT have shown the largest shift, from 1293 cm⁻¹ in F-SWNT to 1304 cm⁻¹, as seen in FIG. 25D. Only a slightly smaller upshift (to 1302 cm⁻¹) was observed for U-F-SWNTs (FIG. 25B), while T-F-SWNTs have shown the smallest shift, to 1297 cm⁻¹ (FIG. 25C). The latter probably indicates that in T-F-SWNTs attachment to the sidewall occurs through the element of different nature (namely sulfur) than that in U-F-SWNTs and G-F-SWNTs which are both linked through the C—N bonds to the sidewall carbons. Thus, the Raman spectrum of T-F-SWNTs supports the covalent bonding of thiourea to F-SWNTs (Scheme 2) primarily through the C—S and not C—N bond.

X-ray photoelectron spectroscopy (XPS). The XPS analysis was done on SWNT products obtained under variable reaction conditions using both reaction schemes (Schemes 2, 3). The elemental analysis data are summarized in Table 1.

TABLE 1
XPS elemental analysis data (at. %) of the derivatized F-SWNT
products obtained under different reaction conditions

| | Temp, | Time, | XPS | XPS | XPS | XPS | XPS |
|---|---|---|---|---|---|---|---|
| Product | ° C. | hours | C1s | F1s | O1s | S2p | N1s |
| F-SWNT | | | 62.6 | 37.4 | | | |
| U-F-SWNT | | | | | | | |
| Solution synthesis | 100 | 4 | 78.6 | 13.2 | 5.5 | | 2.8 |
| Melt synthesis | 150 | 4 | 65.2 | 14.7 | 6.7 | | 13.4 |
| G-F-SWNT | 80 | 4 | 73.4 | 24.1 | | | 2.5 |
| | 100 | 4 | 89.9 | 7.7 | | | 2.4 |
| T-F-SWNT | 80 | 4 | 83.3 | 14.5 | | 0.7 | 1.5 |
| | 100 | 4 | 87.8 | 10.2 | | 0.7 | 1.3 |

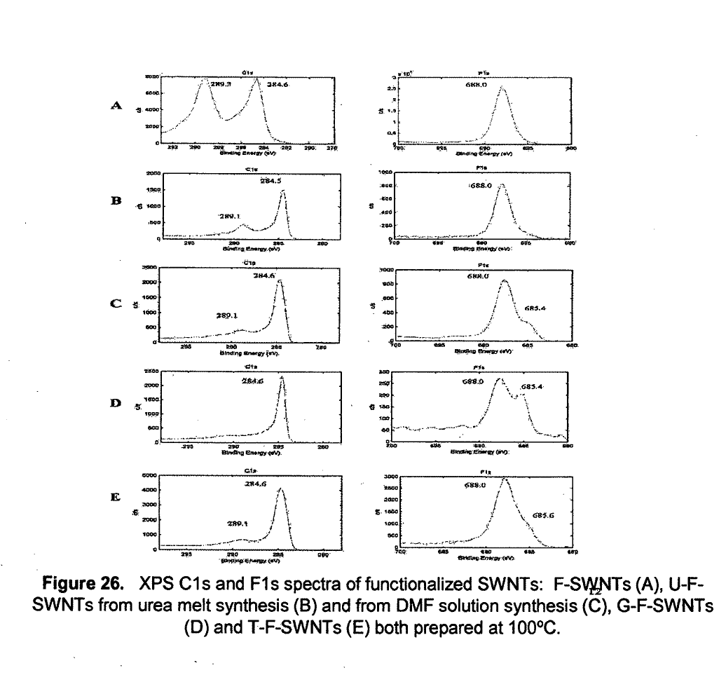

The high-resolution XPS C1s and F1s spectra of functionalized SWNTs are shown on FIG. 26. These data provide information on the extent of fluorine removal from F-SWNTs both through displacement by urea, guanidine and thiourea groups and defluorination reactions. The degree of functionalization of SWNTs can also be estimated from these data. The atomic content of fluorine in F-SWNTs was found to be 37.4 at. % based upon integration of F1s and C1s peaks. All derivatized F-SWNTs have shown the reduced content of fluorine. The most notable change in fluorine content with respect to F-SWNTs was found for the G-F-SWNTs (7.7 at. %) prepared through the reaction (Scheme 2) run at 100° C., while at 80° C. the same reaction yielded the product with F content as high as 24.1 at. %. However, the nitrogen content in the G-F-SWNT derivatives was found not to depend on reaction temperature and remain at about the same level, 2.4-2.5 at. %. The degree of sidewall functionalization (R/C) by guanidine groups was estimated from elemental analysis data (after deduction of atomic percent of carbons bonded to fluorine) and found to decrease with the reaction temperature, from 1:58 to 1:100. This is most likely related to guanidine's high basicity which facilitates predominant occurrence of defluorination other than nucleophilic substitution of fluorine in F-SWNTs at higher temperatures.

Somewhat similar trend was observed for T-F-SWNTs. XPS data show more fluorine removal from F-SWNTs with the reaction temperature increase and virtually no change in the content of sulfur and nitrogen (Table 1). The measured S/N atomic ratio of 1:2 in the reaction product (Scheme 3) supports the attachment of thiourea molecules which are estimated to bond to about 1 in 90 carbons on the SWNT sidewall.

For U-F-SWNTs, prepared by urea melt synthesis, XPS analysis yielded a much higher content of nitrogen (13.4 at. %) as compared to only 2.8 at. % content found in the product prepared through the DMF solution synthesis. The former has also shown an accurate (2:1) nitrogen to oxygen atomic ratio, as expected from stoichiometry of the attached urea groups, while the latter demonstrated a considerably elevated content of oxygen in relation to nitrogen (Table 1), which can be related to the presence of {-NC(═O)O—}ₓ units in the PolyU-F-SWNT byproduct formed in DMF under solvothermal synthesis conditions. The XPS elemental analysis data suggest that the urea melt synthesis yields the F-SWNT derivative having the degree of sidewall functionalization by urea molecules as high as 1 in 8 carbons.

In FIG. 26A, the high-resolution XPS spectrum of F-SWNTs shows a C1s peak with maxima at 284.6 and 289.3 eV due to the C═C and C—F bonded carbons. In the C1s spectra of derivatized F-SWNTs (FIG. 326B-E) the peak of the C—F bonded carbons at 289.1 eV is significantly decreased in intensity for each derivative indicating that the amount of the bonded fluorine is diminished with functionalization. The position of C1s peak at 289.1 eV reflects the predominantly covalent nature of the C—F bond in the F-SWNTs and their derivatives since this peak is located very close to the C—F carbon peak position in the spectra of fluorographite C₂F. This is also confirmed by the observed position of F1s peak at 688.0 eV in the XPS spectra of F-SWNTs (FIG. 26A) and all studied derivatives (FIGS. 26B-E), where this peak is located only slightly below the maximum value for the covalent C—F bond in PTFE (689 eV).

It should be noted that in the XPS F1s spectra of U-F-SWNTs, G-F-SWNTs and T-F-SWNTs, which are all prepared through DMF solution synthesis, an additional shoulder peak at 685.4 eV has appeared. The position of this peak suggests the presence of ionic fluorine most likely from HF which is the reaction byproduct (Schemes 2, 3) capable of forming salt with the F-SWNT amide and heteroamide derivatives. The shoulder peak at 685.4 eV shown by G-F-SWNT (FIG. 26D) has an increased intensity compared to other nanotube derivatives due to higher basicity of guanidine moieties as compared to urea and thiourea. It should be pointed out that the peak due to ionic fluorine does not appear in the XPS F1s spectrum of U-F-SWNTs (FIG. 26B) produced at 150° C. under solvent-free urea melt process conditions when HF entirely evaporates.

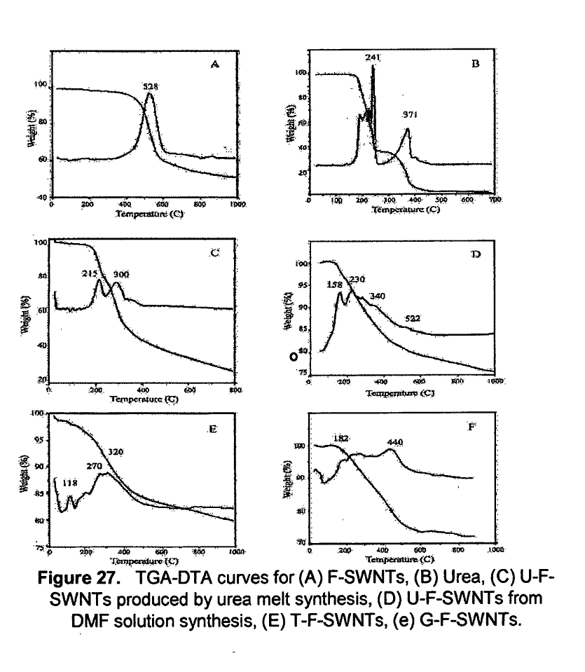

Thermal gravimetric analysis (TGA). Thermal degradation studies were carried out in argon flow environment under continuous heating at 10° C./min up to 1000° C. The differential weight curve of the F-SWNT precursor, shown in FIG. 27A, displays a single degradation peak at 528° C. due to the removal of fluorine which is known to form CF₄ as a major degradation product. The TGA residue of 51 wt. % was confirmed to be SWNTs that were defluorinated as evidenced by the decreased D peak and increased G peak intensities in the Raman spectrum (not shown). Urea itself was also subjected to TGA, and it was found that there is a two-step degradation curve for urea, showing two major peaks on DTA plot, one at 241° C. and the second at 371° C. (FIG. 27B). These peaks are most likely due to well-known decomposition of urea into ammonia and isocyanic acid HNCO. The TGA-DTA curves for the U-F-SWNTs (FIG. 27C-D) also show a degradation occurring in the 200-350° C. temperature range with the DTA peaks shifted relatively to urea itself. This indicates that urea moieties are attached to the SWNTs by covalent sidewall C—N bonds which cleave in U-F-SWNTs in the same temperature region as sidewall amino functionalized SWNTs. It was also found that DTA curve virtually lacked a peak at 400-600° C., confirming that most of the fluorine was removed from the F-SWNT sidewall. Based on weight loss, it was estimated that about 1 in 8 carbons on the U-F-SWNT sidewall are functionalized with urea molecules by melt synthesis, which is in close agreement with the estimation from XPS analysis data. However, this should be considered as an upper limit for the degree of sidewall functionalization by urea since PolyU-F-SWNT by-product can also be present in the sample and contribute into weight loss during TGA.

The TGA curves for all three F-SWNT derivatives prepared by DMF solution synthesis at 80° C. (FIG. 27D-F) show significantly lower total weight loss (20-30%) as compared to urea melt synthesized U-F-SWNTs (˜70%). This should reflect a smaller number of amide and heteroamide functionalities attached to the SWNTs. The DTA curves for these products (FIGS. 27D-F) exhibit an additional peak at 100-190° C. which can be explained by release of HF from salts formed by amide groups. By taking into account only the weight loss occurring in the 200-400° C. temperature range due to detachment of covalently bonded groups, the degree of sidewall functionalization by DMF solution synthesis can be estimated as approximately 1 in 25 for U-F-SWNTs, 1 in 45 for T-F-SWNTs, and 1 in 20 for G-F-SWNTs. The discrepancy of these numbers with the XPS based estimation is related to a difficulty in accurately quantifying the weight loss due to residual covalently bonded fluorine on F-SWNT derivatives.

Scanning electron microscopy (SEM). The SEM studies helped to reveal the surface morphology and extent of nanotube bundling within bulk nanotube samples. The SEM image of the U-F-SWNTs from melt synthesis given as an example on FIG. 13, shows a very thin nanotube bundles. It is interesting to note that the SEM investigation of these U-F-SWNTs has exposed their modified electrical properties. Usually, F-SWNTs have a high resistivity, and they must be made conductive for SEM imaging by coating with gold. In case of U-F-SWNTs, we found no need to coat the sample surface with gold as clear images were obtained even at a magnification of 120,000×. This indicates that the increased conductivity of F-SWNTs results from their surface modification through urea functionalization. The modified electrical properties of U-F-SWNTs and other derivatives are currently under investigation.

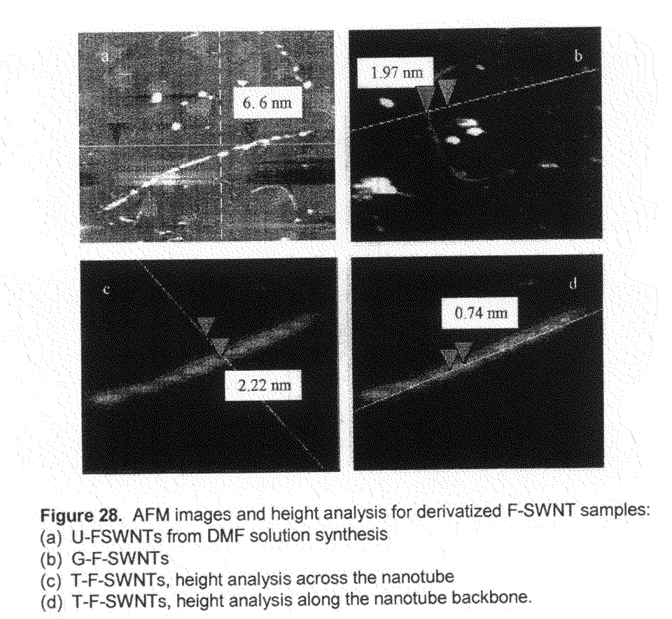

Atomic force microscopy. AFM studies have provided direct evidence for surface modification in derivatized F-SWNTs. The AFM image of the specimen from U-F-SWNTs (FIG. 28a), which were prepared from F-SWNTs through a DMF solution synthesis, shows small and large beads on the backbones of some nanotubes. From the cross-section height analysis indicated by the flags in FIG. 28a the size of the nanotube with the sidewall-attached beads was estimated to be 6.6 nm. Note that there are different size beads along the backbone of this and some other nanotubes seen on the image. The beads are most likely the result of polyurea formation on the nanotubes producing PolyU-F-SWNT by-product. The same AFM image (FIG. 28a) shows that there are also many shorter length nanotubes without beads on the sidewalls present in the sample.

At the same time, none of the zoomed AFM images of G-F-SWNT (FIG. 28b) and T-F-SWNT (FIG. 28c-d) samples shows beads on the nanotubes indicating that polymerization reactions most likely did not occur during the reaction of F-SWNTs with guanidine and thiourea. The tapping mode analysis of the cross-section profile of single G-F-SWNT nanotube shows the height of 1.97 nm (FIG. 28b) which after deduction of the mean diameter value of the nanotube frame (˜1.2-1.4 nm) yields the expected size of guanidine moiety covalently attached to the sidewall. The height analysis of the T-F-SWNT single nanotube sample yields a 2.22 nm height across the derivatized nanotube (FIG. 28c) and 0.74 nm difference measured along the backbone area (FIG. 28d). The latter value represents the approximate length of the —S—C(═NH)NH₂ group attached to the nanotube sidewalls in a “stretched” fashion.

Transmission electron microscopy (TEM). Although nanotubes decorated with covalently attached beads of polymerized urea were clearly observed in the AFM images, it appears that no large beads on the nanotubes are seen in our TEM images. This can be accounted for by the differences in the procedure of sample preparation for SEM and TEM. The samples for TEM studies are prepared from the functionalized nanotubes after their re-suspension by sonication followed by centrifugation of the suspension and sampling of the top part of the suspension. The presence of many nanotubes still in the form of bundles should be noted. The images of U-F-SWNTs are shown in FIGS. 18-21 at different magnifications. The presence of more single nanotubes than larger bundles in the TEM sample is another indication of strong intercalating nature of urea into the larger bundles to produce smaller bundles and singles.

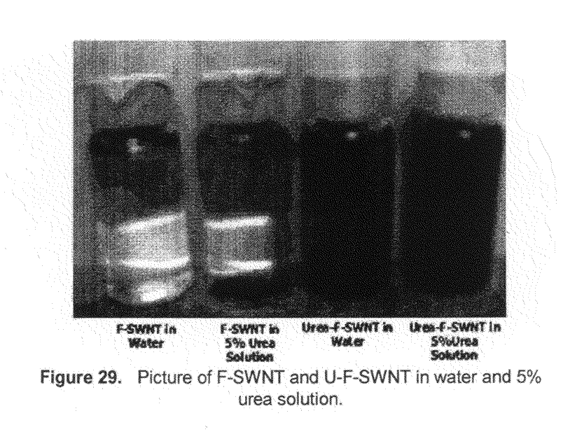

Dispersion in solvents. To study the effect of functional groups on the nanotubes on dispersion ability in different solvent systems, 5 mg of F-SWNTs and U-F-SWNTs were placed into a 20 mL vial containing either pure de-ionized water or 5 wt. % urea in water solution. The vials were placed into a bath sonicator and sonicated for 15 minutes. The obtained suspensions were let standing for about one hour, then the photographs were taken. As shown on pictures in FIG. 29, the F-SWNTs, due to their hydrophobic nature, do not disperse and remain on top of water. However, when placed in the 5% urea solution, the F-SWNTs seem to enlarge in volume and migrate to the bottom of the vial. This indicates that even though urea is extremely soluble in water, it still can be drawn to the Van der Waals forces within the F-SWNT bundles, intercalate, wrap and thus separate them, which should result in an enlarged, “swelled” appearance of the sample. It also seems that the hydrophobic nature of the fluorinated tubes has been somewhat overcome by creating a hydrophilic coating by urea over the bundles which under the weight of the coating sank to the bottom of the vial.

U-F-SWNTs show a much better dispersion in water compared to F-SWNTs, as the solution visibly remains homogeneous and dark, exhibiting only a small amount of “swelled” nanotube precipitate on the bottom of the vial (FIG. 29). Finally, the U-F-SWNTs in the 5% urea solution produced the best dispersion, as the vial was entirely dark. This dispersion has shown no precipitate even after many months. This is an important result, as it could enable uses of U-F-SWNTs for biomedical research carried out mostly in an aqueous environments.

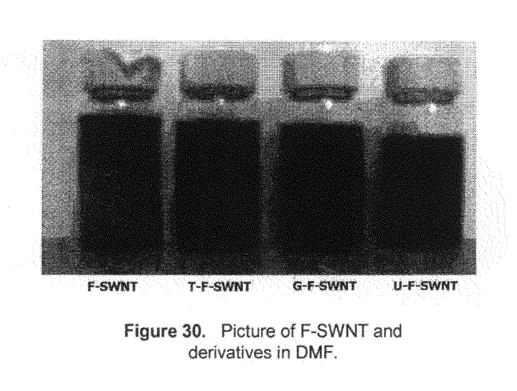

In comparison, T-F-SWNTs and G-F-SWNTs did not form stable suspensions in water. These derivatives, as well as U-F-SWNTs, however, dispersed well in DMF and showed no or little precipitation after many weeks of standing with U-F-SWNTs forming the darkest colored solution, as seen on FIG. 30. For the F-SWNT suspension, the dispersion was observed not to be very dark and partial precipitation from DMF was seen on the bottom of the vial.

From the synthetic chemistry point of view, the development of solvent-free one-step urea melt synthesis will add to the number of green chemistry methods of functionalizing nanotubes. The demonstrated methods help to create bifunctionalized nanotubes in a facile manner. From the applications standpoint, electrical resistivity measurements are in progress for all derivatives. The new derivatives with their amide terminal groups may be useful in nanotube-FET devices. As it relates to the present disclosure, U-F-SWNTs, T-F-SWNTs and G-F-SWNTs have potential as mechanical strength reinforcers in epoxy composites. The ability to disperse these functionalize SWNTs in aqueous systems may help generate new research and biological tools.

Finally, in some embodiments the present disclosure provides substituted carbon nanotubes generated from F-SWNTs that have been reacted with compounds of formula II:

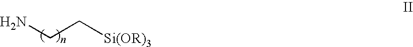

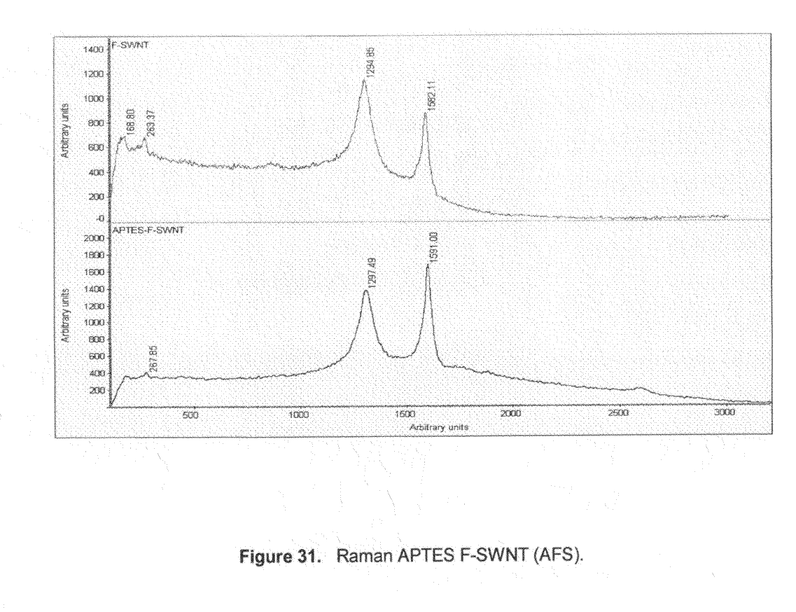

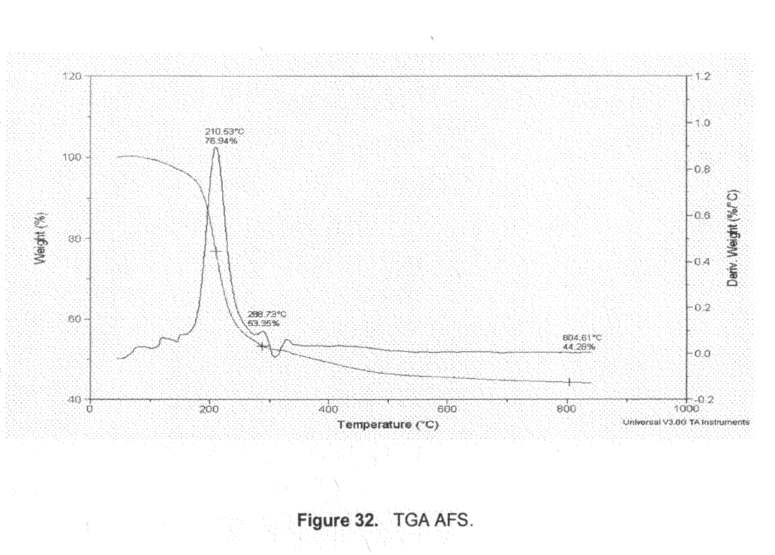

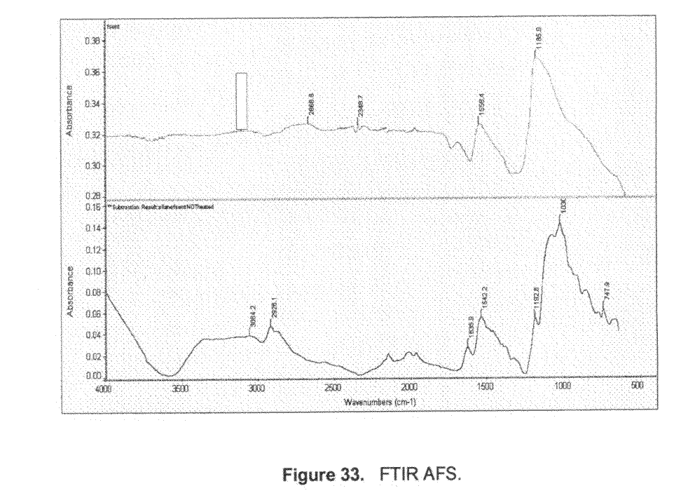

In this general formula n is an integer from 0 to 10 and R is an optionally-substituted alkyl group, as describe above. Covalent attachment is accomplished through the amino functional group of the compound of formula II by displacement of fluorine from the F-SWNT. FIGS. 31-33 show the characterization of F-SWNTs reacted with NH₂(CH₂)₃Si(OEt)₃ (APTES), as an exemplary embodiment. FIG. 31 shows a comparison of the Raman spectrum of APTES-F-SWNT with the parent F-SWNT. FIG. 32 shows the TGA analysis of APTES-F-SWNT. FIG. 33 shows the FTIR of APTES-F-SWNT.

Ceramic materials and surface coatings may incorporate these substituted carbon nanotube. Ceramic materials may include, but are not limited to, barium titanate (which may be mixed with strontium titanate), bismuth strontium calcium copper oxide, boron carbide (B₄C). boron nitride. ferrite (Fe₃O₄), lead zirconate titanate, magnesium diboride (MgB₂), silicon carbide (SiC), silicon nitride (Si₃N₄), steatite, uranium oxide (UO₂), yttrium barium copper oxide (YBa₂Cu₃O₇₋ₓ.), zinc oxide (ZnO), and zirconium dioxide (zirconia).

The silane portion may be used to form a coating on glasses, for example, silicon oxide type surfaces. Incorporation of these functionalized SWNTs into other oxide coatings such as ITO films in solar cell devices and the like may also prove beneficial.

In summary, the present invention provides mechanically-reinforced polymer composites loaded with long chain alkyl- and urea-functionalized carbon nanotubes. The functionalization of fluorinated carbon nanotubes with long chain alkyl and/or urea groups improves the dispersion of nanotubes in polymer matrices and creates a suitable interface due to a covalent bonding of nanotubes to a polymer matrix. The possible applications of these mechanically reinforced polymer composites are for making a strong light-weight materials for airplanes, ships, cars, sporting goods, gas storage containers, etc. As described herein the use of bi-functionalized nanotubes, such as long chain alkylated-fluorinated SWNTs and urea-fluorinated SWNTs, where one or both functional groups assist first in exfoliation of SWNT bundles, and then in dispersion in MDPE during melt processing by shear mixing, facilitating a more efficient interaction and in-situ covalent bonding of SWNT sidewalls to a polymer matrix.

What is claimed:

1. A polymer composite comprising:
a polymer matrix; and
an alkyl-substituted carbon nanotube.

2. The composite of claim 1, wherein the polymer matrix is chosen from thermosets and thermoplastics.

3. The composite of claim 2, wherein the thermoset is chosen from phenol formaldehyde resin, epoxy resin, melamine resin, vulcanized rubber, and polyester resin.

4. The composite of claim 2, wherein the thermoplastic is chosen from acrylonitrile butadiene styrene (ABS), celluloid, cellulose acetate, ethylene-vinyl acetate (EVA), ethylene vinyl alcohol (EVAL), fluorinated ethylene-propylene (FEP), perfluoroalkoxy polymer resin (PFA), chlorotrifluoroethylene (CTFE), ethylene chlorotrifluoroethlyene (ECTFE), ethylene tetrafluoroethylene (ETFE), polyacetal (POM), polyacrylates, polyacrylonitrile (PAN), polyamide (PA), polyamide-imide (PAD, polyaryletherketone (PAEK), polybutadiene (PBD), polybutylene (PB), polybutylene terephthalate (PBT), polyethylene terephthalate (PET), polycyclohexylene dimethylene terephthalate (PCT), polycarbonate (PC), polyhydroxyalkanoates (PHAs), polyketone (PK), polyester, polyethylene (PE), polyetheretherketone (PEEK), polyetherimide (PEI), polyethersulfone (PES, polyethylenechlorinates (PEC), polyimide (PI), polylactic acid (PLA), polymethylpentene (PMP), polyphenylene oxide (PPO), polyphenylene sulfide (PPS), polyphthalamide (PPA), polypropylene (PP), polystyrene (PS), polysulfone (PSU), polyvinyl chloride (PVC), and polyvinylidene chloride (PVDC).

5. The composite of claim 1, wherein the alkyl-substituted carbon nanotube is further fluorinated.

6. The composite of claim 1, wherein alkyl groups of the alkyl-substituted carbon nanotube are optionally-substituted with a functional group capable of forming a covalent link within the polymer matrix.

7. The composite of claim 6, wherein the functional group is chosen from alkenes, alcohols, amines, carboxylic acids, amides, and thiols.

8. The composite of claim 1, wherein the alkyl-substituted carbon nanotube is dispersed in the polymer matrix by extrusion.

9. A polymer composite comprising:
a polymer matrix; and
a fluorinated carbon nanotube reacted with a compound of formula I: 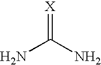
wherein X is selected from the group consisting of O, S, and NH.

10. The composite of claim 9, wherein the polymer matrix is chosen from thermosets and thermoplastics.

11. The composite of claim 10, wherein the thermoset is chosen from phenol formaldehyde resin, epoxy resin, melamine resin, vulcanized rubber, and polyester resin.

12. The composite of claim 10, wherein the thermoplastic is chosen from acrylonitrile butadiene styrene (ABS), celluloid, cellulose acetate, ethylene-vinyl acetate (EVA), ethylene vinyl alcohol (EVAL), fluorinated ethylene-propylene (FEP), perfluoroalkoxy polymer resin (PFA), chlorotrifluoroethylene (CTFE), ethylene chlorotrifluoroethlyene (ECTFE), ethylene tetrafluoroethylene (ETFE), polyacetal (POM), polyacrylates, polyacrylonitrile (PAN), polyamide (PA), polyamide-imide (PAI), polyaryletherketone (PAEK), polybutadiene (PBD), polybutylene (PB), polybutylene terephthalate (PBT), polyethylene terephthalate (PET), polycyclohexylene dimethylene terephthalate (PCT), polycarbonate (PC), polyhydroxyalkanoates (PHAs), polyketone (PK), polyester, polyethylene (PE), polyetheretherketone (PEEK), polyetherimide (PEI), polyethersulfone (PES, polyethylenechlorinates (PEC), polyimide (PI), polylactic acid (PLA), polymethylpentene (PMP), polyphenylene oxide (PPO), polyphenylene sulfide (PPS), polyphthalamide (PPA), polypropylene (PP), polystyrene (PS), polysulfone (PSU), polyvinyl chloride (PVC), and polyvinylidene chloride (PVDC).

13. The composite of claim 9, wherein the fluorinated carbon nanotube forms a covalent link within the polymer matrix.

14. The composite of claim 9, wherein the fluorinated carbon nanotube is dispersed in the polymer matrix by extrusion.

15. A method of functionalizing a carbon nanotube comprising heating a fluorinated carbon nanotube with a compound of formula I: 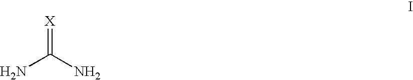
wherein X is selected from the group consisting of O, S, and NH.

16. The method of claim 15, wherein the step of heating comprises melting urea (X═O) in the absence of solvent.

17. The method of claim 15, further comprising sonicating a DMF suspension of the fluorinated carbon nanotubes prior to the step of heating.

18. A functionalized carbon nanotube made by the process of claim 15.

19. A substituted carbon nanotube comprising:
a fluorinated carbon nanotube; and
a compound of formula II: 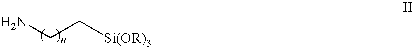
wherein n is an integer from 0 to 10; and
R is an optionally-substituted alkyl group; and
wherein the compound of formula II is covalently attached to the fluorinated nanotube through the amino functional group.

20. A ceramic comprising the substituted carbon nanotube of claim 19.

21. A surface coating comprising the substituted carbon nanotube of claim 19.
